(19) United States
(12) Patent Application Publication
(10) Pub. No.: US 2015/0374409 A1
(43) Pub. Date: Dec. 31, 2015

(54) Dermabrasion Device

(71) Applicant: RECKITT BENCKISER (BRANDS) LIMITED, Slough, Berkshire (GB)

(72) Inventors: Mohammed Chopdat, Hull (GB); Darren Morgan, Hull (GB); Victoria Perrin, Hull (GB); Michael Liang, Dongguan (CN); Richard Clough, Warwick (GB)

(21) Appl. No.: 14/762,531

(22) PCT Filed: Feb. 4, 2014
(86) PCT No.: PCT/GB2014/050304

§ 371 Date: Jul. 22, 2015

(30) Foreign Application Priority Data
Feb. 4, 2013 (GB) 1301940.1

(51) Int. Cl. A61B 17/54

(52) U.S. Cl. CPC A61B 17/54

(57) ABSTRACT

The present invention is directed to a dermabrasion device (1) comprising a handle portion (3), a head portion (2) having two arms (4, 5) arranged to receive and hold a drum (6) having an abrasive surface wherein the abrasive surface is in the form of a convex curve such that the diameter at the centre of the drum (6) is greater than the diameter of the ends of the drum (6).

The present invention relates to a dermabrasion device; and, more particularly, to an electrical dermabrasion device for removing a hard, dry skin layer on a surface of the foot, for example, the heel, the sole or the toes.

Calluses and/or areas of hard skin occur on feet and can be caused by persistent rubbing or uneven pressure, for example from ill-fitting shoes. They are most commonly found at the heel, the ball of the foot and the sides of the toes. Calluses are often unsightly, and the thicker they are the more yellow they can look. With time, particularly thick calluses can become cracked and painful.

There are a number of known methods for reducing or removing calluses and hard skin, generally based upon rubbing, scraping or cutting the hardened skin away. In many cases this may include a first step of softening the skin by soaking the feet or hands in water or by applying some form of softening lotion to the hard skin. The hard skin can be reduced or removed using a device with an abrasive surface, such as a pumice stone, emery board or a device with carborundum paper attached to it. These devices can be manual or electrically operated and in the latter case, an abrasive head can be vibrated and rotated over the hard skin. Examples of such devices are described in WO03/022175, or WO 2012/120373.

The known abrasive devices have a number of drawbacks. In many cases they are unable to conform to the curvature of the parts of the hands or feet where calluses usually occur or are designed in such a way that the surface area available for treatment of the surface of the foot is low/unsatisfactory.

The present application provides a dermabrasion device that provides more optimal efficiency than these devices.

According to a first aspect of the present invention there is provided a dermabrasion device comprising a handle portion, a head portion having two arms arranged to receive and hold a drum having an abrasive surface wherein the abrasive surface is in the form of a convex curve such that the diameter at the centre of the drum is greater than the diameter of the ends of the drum.

Typically, the surface of the drum is in the form of a parabolic arc. Typically the length of the drum is between 2 and 4 times greater than the diameter of the drum. More typically the length of the drum is between 2 and 3 times greater than the diameter of the drum. Most typically, the length of the drum is 2.5 times greater than the diameter of the drum. Preferably, the drum is in the shape of a barrel. A more preferred shape is that of an elongated barrel.

Preferably, the diameter at the centre of the drum is between 10% and 30% larger than the diameter at the ends of the drum. More preferably, the diameter at the centre of the drum is between 15% and 25% larger than the diameter at the ends of the drum. More preferably, the diameter at the centre of the drum is between 20% and 22% larger than the diameter at the ends of the drum.

Typically, at least 80% of the surface area of the drum is used in the dermabrasion process. More typically, at least 85% of the surface area of the drum is used in the dermabrasion process. Even more typically, at least 90% of the surface area of the drum is used in the dermabrasion process.

Typically the abrasive surface of the drum is not provided with an antimicrobial agent.

The material which forms the abrasive surface of the drum can be selected to provide the desired level of abrasiveness. For example, the abrasive surface can be made of a coarse material if a high level of abrasiveness is required, or can be made of a fine material if a low level of abrasiveness is required.

The material which forms the abrasive surface can be provided with additional abrasive materials such as diamond fragments or pumice.

According to a second aspect of the present invention there is provided a dermabrasion device comprising a handle portion, a head portion having two arms arranged to receive and hold a drum having an abrasive surface wherein the abrasive surface is in the form of a concave curve such that the diameter at the centre of the drum is smaller than the diameter of the ends of the drum.

Typically, the surface of the drum is in the form of a parabolic concave arc. Typically the length of the drum is between 2 and 3 times greater than the diameter of the drum.

More typically, the length of the drum is 2.7-2.8 times greater than the diameter of the drum. A preferred shape is that of a concave barrel.

Preferably, the diameter at the centre of the drum is between 10% and 30% smaller than the diameter at the ends of the drum. More preferably, the diameter at the centre of the drum is between 15% and 25% smaller than the diameter at the ends of the drum. More preferably, the diameter at the centre of the drum is between 17% and 20% smaller than the diameter at the ends of the drum.

Typically, at least 80% of the surface area of the drum is used in the dermabrasion process. More typically, at least 85% of the surface area of the drum is used in the dermabrasion process. Even more typically, at least 90% of the surface area of the drum is used in the dermabrasion process.

Typically the abrasive surface of the drum is not provided with an antimicrobial agent.

The material which forms the abrasive surface of the drum can be selected to provide the desired level of abrasiveness. For example, the abrasive surface can be made of a coarse material if a high level of abrasiveness is required, or can be made of a fine material if a low level of abrasiveness is required.

The material which forms the abrasive surface can be provided with additional abrasive materials such as diamond fragments or pumice.

In alternative embodiments of the device of the first and second aspects of the present invention the drum can be provided with a material such as corrugated rubber which provides a massaging effect instead of an abrasive surface. The corrugated rubber can be made of a silicone rubber or thermoplastic elastomer (TPE). Alternatively, the massaging material can be made of a sponge.

An example embodiment of the present invention will now be described in more detail with reference to the accompanying Figures, in which:

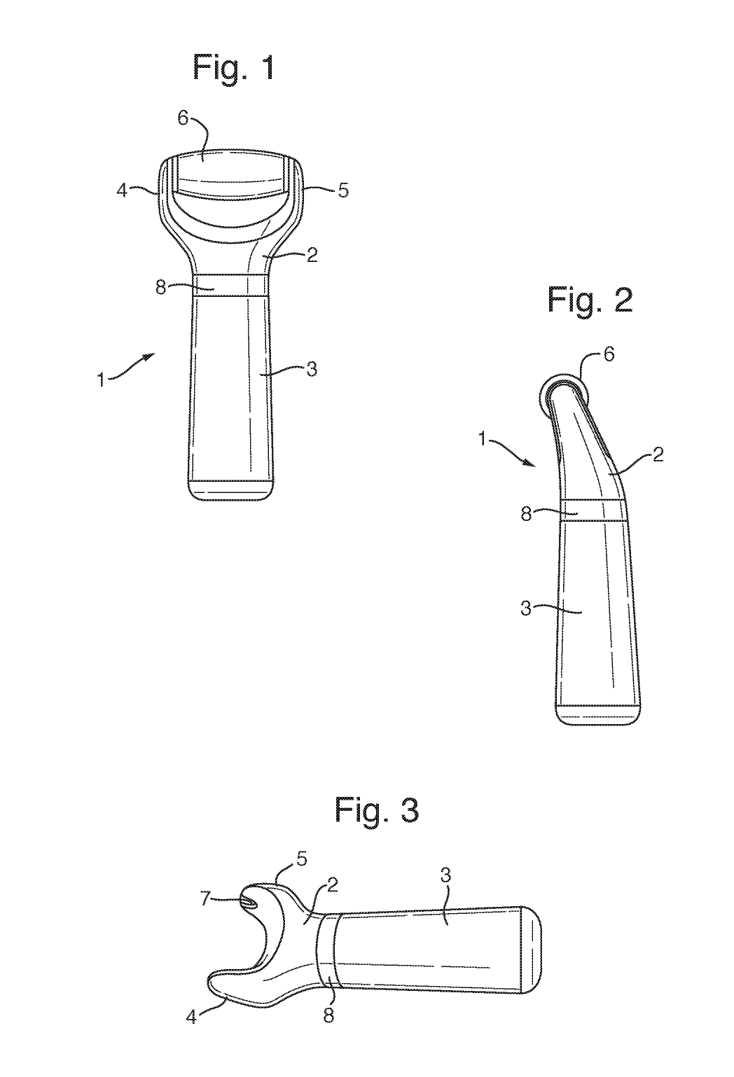

FIG. 1 illustrates a front view of a dermabrasion device in accordance with the present invention;

FIG. 2 illustrates a side view of a dermabrasion device in accordance with the present invention; and

FIG. 3 illustrates a dermabrasion device in accordance with the present invention with the abrasion drum removed.

In the Figures there is generally depicted a dermabrasion device at 1. The device 1 comprises a head 2 and a handle 3. The head 2 is provided with arms 4 and 5. A drum 6 is attached to and held by the arms 4 and 5 in such a way that the drum 6 can rotate along its longitudinal axis at a sufficient rate to achieve dermabrasion. The ends of drum 6 are provided with an axis or shaft (not shown). The axis or shaft at each end of drum 6 connects with an aperture 7 in each arm 4 and 5. The drum 6 is driven by an electro-mechanical mechanism (not shown), such as an electric motor, which are well-known to the man skilled in the art. The electro-mechanical mechanism is activated by a switch 8.

From FIG. 2 it can be seen that in one embodiment the head 2 is angled in such a way as to make it easier to position the drum 6 against the skin to achieve dermabrasion.

The drum 6 can be released from the head 2 using a switch (not shown) suitably located in one of the arms 4 and 5.

In use, the device is activated by switch 8 which causes the drum 6 to rotate. The surface of the drum 6 is provided with an abrasive material. The abrasive material causes the hard skin or callus to be removed efficiently. The curvature of the surface of the drum 6 allows a user greater freedom in abrading the skin by providing a greater surface area for contact and also improved conformance/connectivity with different areas of the skin.

An advantage of the present invention is that there is provided a dermabrasion device which has a working surface area, i.e. a surface area available for contact with the skin, that is significantly greater than the working surface area of devices that are currently available.

A further advantage of the shape of the dermabrasion drum is that any stress on the drum is more evenly distributed across its surface. This removes the need for any additional supporting structures which increase the complexity of the device and reduce the working surface area.

Further modifications and developments can be made without departing from the scope of the invention described herein.

1. A dermabrasion device comprising:
a handle portion;
a head portion having two arms arranged to receive and hold a drum having an abrasive surface;
wherein the abrasive surface is in the form of a convex curve such that the diameter at the center of the drum is greater than the diameter of the ends of the drum.

2. The dermabrasion device as claimed in claim 1, wherein the surface of the drum is in the form of a parabolic arc.

3. The dermabrasion device as claimed in claim 1, wherein the length of the drum is between 2 and 3 times greater than the diameter of the drum.

4. The dermabrasion device as claimed in claim 3, wherein the length of the drum is 2.5 times greater than the diameter of the drum.

5. The dermabrasion device as claimed in claim 1, wherein the drum is in the shape of a barrel.

6. The dermabrasion device as claimed in claim 5, wherein the drum is in the shape of an elongated barrel.

7. The dermabrasion device as claimed in claim 1 wherein the diameter at the center of the drum is between 10% and 30% larger than the diameter at the ends of the drum.

8. The dermabrasion device as claimed in claim 7, wherein the diameter at the center of the drum is between 15% and 25% larger than the diameter at the ends of the drum.

9. The dermabrasion device as claimed in claim 7, wherein the diameter at the center of the drum is between 20% and 22% larger than the diameter at the ends of the drum.

10. A dermabrasion device comprising:
a handle portion;
a head portion having two arms arranged to receive and hold a drum having an abrasive surface;
wherein the abrasive surface is in the form of a concave curve such that the diameter at the center of the drum is smaller than the diameter of the ends of the drum.

11. The dermabrasion device as claimed in claim 10, wherein the surface of the drum is in the form of a parabolic concave arc.

12. The dermabrasion device as claimed in claim 10, wherein the length of the drum is between 2 and 3 times greater than the diameter of the drum.

13. The dermabrasion device as claimed in claim 12, wherein the length of the drum is 2.7-2.8 times greater than the diameter of the drum.

14. The dermabrasion device as claimed in claim 10, wherein the shape of the drum is that of a concave barrel.

15. The dermabrasion device as claimed in claim 10, wherein the diameter at the center of the drum is between 10% and 30% smaller than the diameter at the ends of the drum.

16. The dermabrasion device as claimed in claim 15, wherein the diameter at the center of the drum is between 15% and 25% smaller than the diameter at the ends of the drum.

17. The dermabrasion device as claimed in claim 16, wherein the diameter at the center of the drum is between 17% and 20% smaller than the diameter at the ends of the drum.

18. The dermabrasion device as claimed in claim 10, wherein at least 80% of the surface area of the drum is configured to be used in a dermabrasion process.

19. The dermabrasion device as claimed in claim 18, wherein at least 85% of the surface area of the drum is configured to be used in a dermabrasion process.

20. The dermabrasion device as claimed in claim 18, wherein at least 90% of the surface area of the drum is configured to be used in a dermabrasion process.

21. The dermabrasion device as claimed in claim 1, wherein the abrasive surface of the drum is not provided with an antimicrobial agent.

22. The dermabrasion device as claimed in claim 1, wherein the material which forms the abrasive surface is provided with an abrasive material selected from the group consisting of diamond fragments and pumice.

23. A dermabrasion device comprising:
a handle portion;
a head portion having two arms arranged to receive and hold a drum having a material which provides a massaging effect;
wherein the surface is in the form of a convex curve such that the diameter at the center of the drum is greater than the diameter of the ends of the drum; and
wherein the material which provides the massaging effect is a corrugated rubber.

24. A dermabrasion device comprising:
a handle portion;
a head portion having two arms arranged to receive and hold a drum having a material which provides a massaging effects;
wherein the surface is in the form of a concave curve such that the diameter at the center of the drum is smaller than the diameter of the ends of the drum; and
wherein the material which provides the massaging effect is a corrugated rubber.

25. The dermabrasion device as claimed in claim 10, wherein the abrasive surface of the drum is not provided with an antimicrobial agent.

26. The dermabrasion device as claimed in claim 10, wherein the material which forms the abrasive surface is provided with an abrasive material selected from the group consisting of diamond fragments and pumice.

27. The dermabrasion device as claimed in claim 23, wherein the corrugated rubber is selected from the group consisting of silicone rubber, a thermoplastic elastomer (TPE), and a sponge material.

28. The dermabrasion device as claimed in claim 24, wherein the corrugated rubber is selected from the group consisting of silicone rubber, a thermoplastic elastomer (TPE), and a sponge material.
